(19) United States
(12) Patent Application Publication
(10) Pub. No.: US 2022/0401103 A1
(43) Pub. Date: Dec. 22, 2022

(54) SURGICAL OCCLUDING DEVICES

(71) Applicant: Covidien LP, Mansfield, MA (US)

(72) Inventors: Patrick D. Mozdzierz, Glastonbury, CT (US); Jeremy G. Fidock, Hamden, CT (US); Mallory A. Pille, Middlebury, CT (US); Olesea Diaz-Chiosa, Naugatuck, CT (US)

(21) Appl. No.: 17/746,147

(22) Filed: May 17, 2022

Related U.S. Application Data
(60) Provisional application No. 63/212,161, filed on Jun. 18, 2021.

(51) Int. Cl. A61B 17/12

(52) U.S. Cl. CPC A61B 17/12013; A61B 2017/00367

(57) ABSTRACT

A surgical occluding device includes a body portion configured to radially surround a tissue lumen. The body portion includes a plurality of segments and a plurality of folds. Each fold of the plurality of folds is defined between adjacent segments of the plurality of segments. Half of the folds of the plurality of folds define acute angles, and half of the folds of the plurality of folds define obtuse angles. The body portion is movable from a first orientation where the acute angle defines a first acute angle, to a second orientation where the acute angle defines a second, smaller acute angle.

### CROSS-REFERENCE TO RELATED APPLICATION

This application claims the benefit of and priority to U.S. Provisional Patent Application No. 63/212,161, filed Jun. 18, 2021, the entire contents of which is incorporated by reference herein.

### BACKGROUND

### Technical Field

The disclosure relates generally to surgical devices for occluding tissue. More particularly, the disclosure relates to a surgical devices and methods for occluding hollow tissue organs to help prepare the tissue for an anastomosis procedure.

### Background of Related Art

Anastomosis is the surgical joining of separate hollow organ sections. Typically, an anastomosis procedure follows surgery in which a diseased or defective section of hollow tissue is removed, and the end sections are occluded or sealed (e.g., stapled via a linear surgical stapler). Depending on the desired anastomosis procedure, the end sections may be joined by either circular, end-to-end, or side-to-side organ reconstruction methods.

Certain anastomosis procedures are performed on portions of tissue that are challenging to access. Likewise, it may be challenging to cut, occlude, and/or seal the end sections of tissue prior to the joining of tissue. For instance, lower anterior resection procedures generally involve sealing and segmentation of the lower colon/rectum. The narrow pelvic structure makes it difficult to position traditional linear stapling devices in the desired location. Further, the geometry of non-linear stapling and cutting instruments often requires the surgical device to be non-laparoscopic.

### SUMMARY

The disclosure relates to a surgical occluding device including a body portion configured to radially surround a tissue lumen. The body portion includes a plurality of segments and a plurality of folds. Each fold of the plurality of folds is defined between two adjacent segments of the plurality of segments. Half of the folds of the plurality of folds define acute angles, and half of the folds of the plurality of folds define obtuse angles. The body portion is movable from a first orientation where the acute angle defines a first acute angle, to a second orientation where the acute angle defines a second, smaller acute angle.

In aspects, the plurality of segments may include ten segments.

In aspects, when the body portion is in the first orientation, the obtuse angle defines a first obtuse angle, and when the body portion is in the second orientation, the obtuse angle may define a second, smaller obtuse angle.

In aspects, the surgical occluding device may include a length of suture engaged with the body portion. In disclosed aspects, the length of suture may be engaged with each segment of the plurality of segments.

In aspects, the plurality of folds may include ten folds.

In aspects, the body portion may form a star shape in its first orientation. In disclosed aspects, the body portion may form a star shape in its second orientation.

The disclosure also relates to a surgical occluding device configured to radially surround a tissue lumen, and includes a proximal band, a distal band, and a plurality of spindles. The proximal band includes a first end and a second end. The distal band includes a first end and a second end. The plurality of spindles interconnects the proximal band and the distal band. The proximal band is movable from a first orientation where the first end of the proximal band is free from contact with the second end of the proximal band to a second orientation where the first end of the proximal band contacts the second end of the proximal band. The distal band is movable from a first orientation where the first end of the distal band is free from contact with the second end of the distal band to a second orientation where the first end of the distal band contacts the second end of the distal band.

In aspects, the first end of the proximal band includes structure that is configured to engage structure on the second end of the proximal band to retain the proximal band in the second orientation. In disclosed aspects, the first end of the distal band includes structure that is configured to engage structure on the second end of the distal band to retain the distal band in the second orientation.

In aspects, the proximal band and the distal band are configured to be rolled into a coiled configuration to fit within a portion of a cannula.

In aspects, the plurality of spindles includes between ten spindles and fourteen spindles.

In aspects, the plurality of spindles includes twelve spindles.

In aspects, each spindle of the plurality of spindles is evenly spaced from adjacent spindles of the plurality of spindles.

The disclosure also relates to a method of occluding a tissue lumen. The method includes positioning a first tissue occluding device around a distal portion of a tissue lumen where the tissue lumen defines a longitudinal axis, positioning a second tissue occluding device around a proximal portion of a tissue lumen, compressing the first tissue occluding device to move the first tissue occluding device from a C-shaped orientation to an O-shaped orientation, compressing the second tissue occluding device to move the second tissue occluding device from a C-shaped orientation to an O-shaped orientation, deflecting a plurality of locations of the first tissue occluding device to move the plurality of locations radially inward, and deflecting a plurality of locations of the second tissue occluding device to move the plurality of locations radially inward.

In aspects, the method may include rotating the first tissue occluding device about the longitudinal axis and may further include rotating the second tissue occluding device about the longitudinal axis. In disclosed aspects, the first tissue occluding device is rotated in a first direction, and the second tissue occluding device is rotated in a second, opposite direction.

In aspects, deflecting the plurality of locations of the first tissue occluding device includes deflecting four locations of the first tissue occluding device.

### DESCRIPTION OF THE DRAWINGS

Various aspects of the disclosure are illustrated herein with reference to the accompanying drawings, wherein:

FIG. 1 is a perspective view of a surgical occluding device positioned around a tissue lumen according to a first aspect of the disclosure;

FIG. 2 is a cross-sectional view of the surgical occluding device of FIG. 1 illustrated in a first orientation positioned around the tissue lumen;

FIG. 3 is a cross-sectional view of the surgical occluding device of FIG. 1 illustrated in a second orientation positioned around the tissue lumen, and illustrating suture passing through the tissue;

FIG. 4 is a cross-sectional view of the surgical occluding device of FIG. 1 illustrated in the first orientation positioned around the tissue lumen which has been sutured;

FIG. 5 is a perspective view of a pair of surgical occluding devices illustrated in a first orientation positioned around a tissue lumen according to a second aspect of the disclosure;

FIG. 6 is a cross-sectional view of the surgical occluding device of FIG. 5 illustrated in a second orientation positioned around the tissue lumen;

FIG. 7 is a cross-sectional view of the surgical occluding device of FIG. 5 illustrated in a third orientation positioned around the tissue lumen;

FIG. 8 is a cross-sectional view of the surgical occluding device of FIG. 5 illustrated in a fourth orientation positioned around the tissue lumen;

FIG. 9 is a cross-sectional view of the surgical occluding device of FIG. 5 illustrated in a fifth orientation depicting its position relative to a portion of the tissue lumen;

FIG. 10 is a perspective view of a surgical occluding device positioned around a tissue lumen according to a third aspect of the disclosure;

FIG. 11 is a perspective view of a surgical trocar including the surgical occluding device of FIG. 10 illustrated in a coiled orientation and positioned within a portion of the surgical trocar;

FIG. 12 is a plan view of the surgical occluding device of FIG. 10 illustrated in an open orientation;

FIG. 13 is a side view of the surgical occluding device of FIG. 10 positioned around the tissue lumen;

FIG. 14 is a side view of the surgical occluding device of FIG. 10 positioned around the tissue lumen and illustrated in a twisted configuration;

FIG. 15 is a side view of the surgical occluding device of FIG. 10 positioned around the tissue lumen, illustrated in a twisted configuration, and illustrated following the cutting of a mid-portion of the surgical occluding device; and

FIG. 16 is an enlarged view of the area of detail indicated in FIG. 15.

### DETAILED DESCRIPTION

Aspects of the disclosed surgical occluding devices will now be described in detail with reference to the drawings wherein like numerals designate identical or corresponding elements in each of the several views. As is common in the art, the term “proximal” refers to that part or component closer to the user or operator, e.g., surgeon or physician, while the term “distal” refers to that part or component farther away from the user.

Surgical occluding devices are typically used to seal or occlude segments of tissue and/or compress the tissue around a trocar prior to connecting two segments of the tissue via an anastomosis procedure. It may be difficult to seal, occlude and/or compress tissue when the tissue is challenging to access. The surgical occluding devices disclosed herein facilitate sealing, occluding and/or compressing the ends of tissue segments laparoscopically, for example.

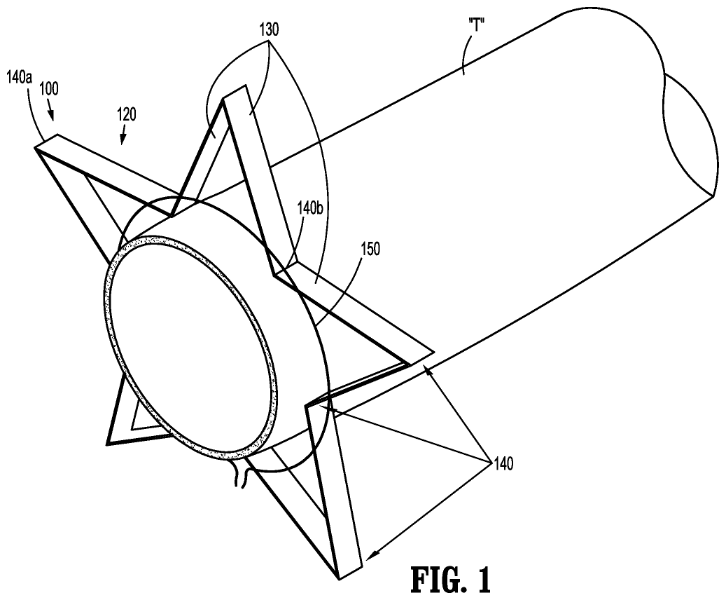

FIGS. 1-4 illustrate an aspect of a surgical occluding device according to the disclosure, referenced generally as numeral 100. The surgical occluding device 100 is illustrated around a tissue lumen “T,” and includes a body portion 120 having a plurality of segments 130. A bend or a fold 140 is defined between two adjacent segments 130. The folds 140 alternate between an acute fold 140a defining an acute angle, and an obtuse fold 140b defining an obtuse angle. As shown, the alternating folds 140 cause the surgical occluding device 100 to be star-shaped. While the surgical occluding device 100 is shown having five acute folds 140a (defining star points) and five obtuse folds 140b , the surgical occluding device 100 may include more or fewer than five acute folds 140a and five obtuse folds 140b without departing from the scope of the disclosure. The surgical occluding device 100 is movable between actuated and unactuated orientations.

Additionally, as shown in FIG. 1, the surgical occluding device 100 may be preloaded with a suture 150 in its unactuated orientation. In such aspects, the suture 150 may be clipped to the surgical occluding device 100. Alternatively, the surgical occluding device 100 may not be preloaded with a suture 150, and the suture 150 can be applied to the tissue lumen “T” and pulled through portions and/or voids (e.g., between adjacent segments 130) of the surgical occluding device 100 after the surgical occluding device 100 is positioned around the tissue lumen “T.” As discussed in further detail below, after the surgical occluding device 100 transitions to the actuated orientation (FIG. 3), the suture 150 is used to create a purse-string to help occlude the tissue lumen “T.”

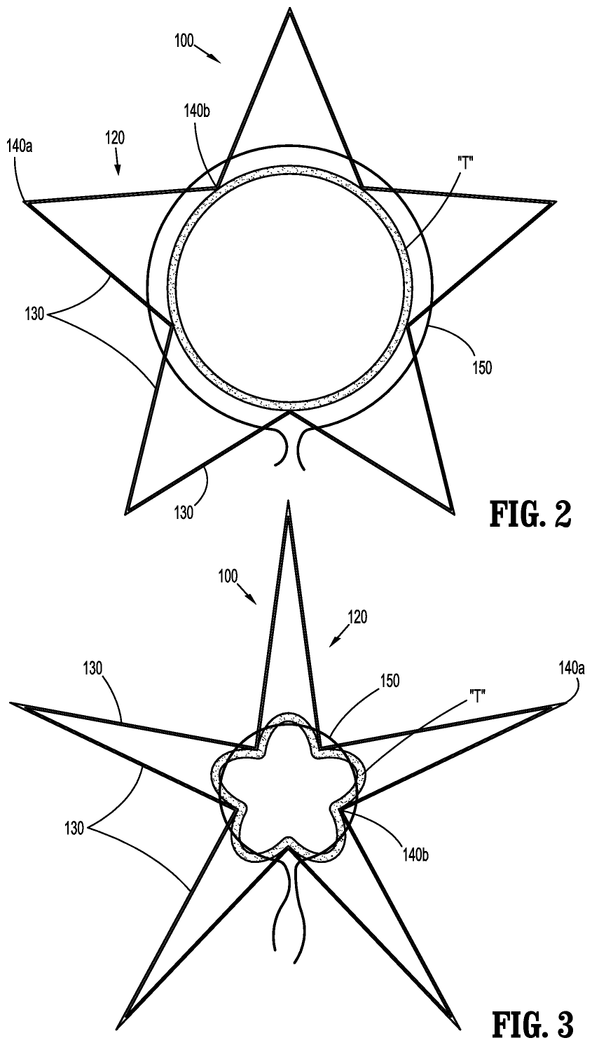

In use, the surgical occluding device 100 is loaded (or preloaded) with suture 150 and is positioned such that the surgical occluding device surrounds a tissue lumen “T” (FIGS. 1 and 2). In aspects, a surgical grasper may be used to position the surgical occluding device 100 in a desired location about the tissue lumen “T.”

Two surgical occluding devices 100 may be used for a single surgical procedure. For instance, one surgical occluding device 100 may be positioned around a distal portion of the tissue lumen “T,” and one surgical occluding device 100 may be positioned around a proximal portion of the tissue lumen “T.”

After the surgical occluding device 100 is positioned around the tissue lumen “T,” a deployment device is used to transition the surgical occluding device 100 from its first, unactuated orientation (FIGS. 1 and 2) to its second, actuated orientation (FIG. 3). In aspects, a suitable deployment device may include a ring having a cam slot, and a plurality of pins (e.g., equal to the number of acute folds 140a of the surgical occluding device 100). A first end of each pin is positionable adjacent an acute fold 140a , and the second end of each pin is engaged with the cam slot of the ring. Here, the pins may be fixed to the ring within a slidable joint to enable radial displacement. The ring is rotated such that the pins are cammed in a manner that causes the first ends of the pins to translate inward towards a central axis defined by the tissue lumen “T,” such that the surgical occluding device 100 is in its second, actuated orientation and is crimped around and compresses the tissue lumen “T,” as shown in FIG. 3

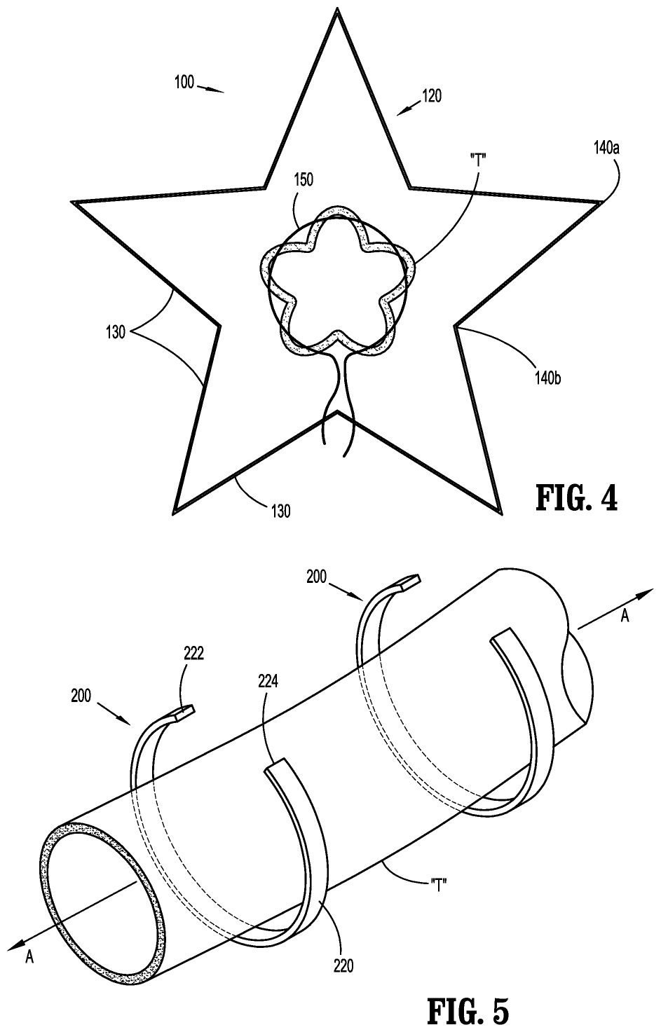

When the surgical occluding device 100 is in its second, actuated orientation, a suturing device (e.g., a laparoscopic needle driver) may be inserted to push or thread the suture 150 through the tissue lumen “T” to create a purse-string (as shown in FIGS. 3 and 4). In aspects, the suturing device is used to create the purse-string while the firing device maintains the surgical occluding device 100 in its second, actuated orientation. Following the suturing of the tissue lumen “T,” the firing device is removed, and the surgical occluding device 100 moves back to its first, unactuated orientation (FIG. 4). In aspects, the surgical occluding device 100 is biased into its first, unactuated orientation. For instance, the surgical occluding 100 may be formed of a resilient material that returns to the non-deformed position. Next, the surgical occluding device 100 may be removed from the surgical site, and an anastomosis procedure may be performed.

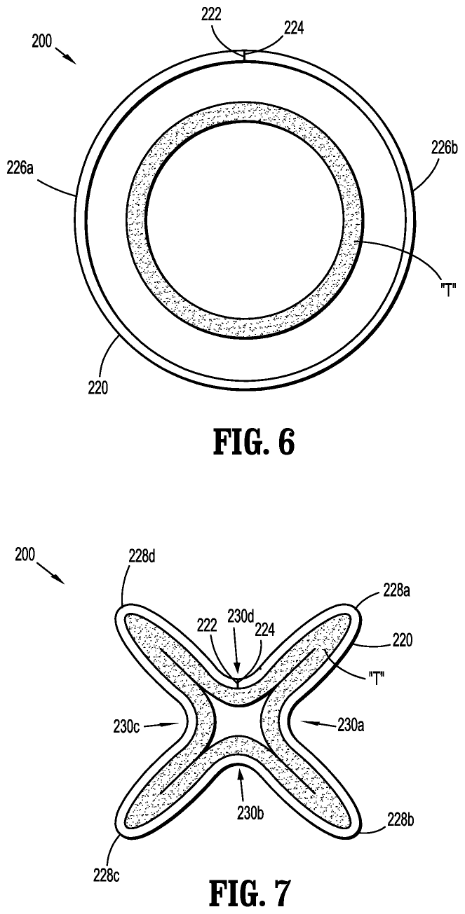

FIGS. 5-9 illustrate a second aspect of a surgical occluding device according to the disclosure, referenced generally as numeral 200. In FIG. 5, two surgical occluding devices 200 are illustrated around a tissue lumen “T,” and each surgical occluding device 200 includes a body portion 220 having a first end 222 and a second end 224. The body portion 220 is movable between a first position where the body portion 220 forms a C-like shape (FIG. 5) where the first end 222 is spaced from the second end 224, and a second position where the body portion 220 forms an O-like shape (FIG. 6) where the first end 222 contacts the second end 224. In aspects, the first end 222 and/or the second end 224 of the body portion 220 includes engagement structure for holding or releasably holding the first end 222 and the second end 224 together.

In aspects, the body portion 220 includes a plurality of deflection locations 230a , 230b , 230c , 230d (FIG. 7) that are configured to deflect (e.g., pre-bent, made of a thinner gauge material, etc.), or an entirety of the body portion 220 is readily deflectable in response to an appropriate application of force. While FIG. 7 illustrates the body portion 220 of the surgical occluding device 200 having been deflected at four deflection locations 230a , 230b , 230c , 230d , the body portion 220 may include more or fewer than four deflection locations without departing from the scope of the disclosure.

In use, two surgical occluding devices 200 are positioned in a spaced-apart manner and surround portions of a tissue lumen “T” (FIG. 5). In aspects, a surgical grasper and/or a deployment device may be used to position the surgical occluding devices 200 in the desired locations about the tissue lumen “T”. For instance, one surgical occluding device 200 may be positioned around a distal portion of the tissue lumen “T,” and one surgical occluding device 200 may be positioned around a proximal portion of the tissue lumen “T.” Additionally, the surgical grasper and/or deployment device may be used to transition the surgical occluding devices 200 from their original C-like orientation (FIG. 5) to the O-like orientation (FIG. 6) by compressing opposing side portions 226a, 226b of the body portion 220 towards each other, for instance.

After the surgical occluding devices 200 are positioned around the tissue lumen “T” and transitioned to their O-like orientation (shown in FIG. 6), a firing device is used to transition the surgical occluding devices 200 from their O-like orientation to a pronged or compressed orientation (FIG. 7) having a plurality of prongs 228a , 228b , 228c , 228d . In aspects, the firing device includes a plurality of arms (e.g., the same number of arms as the number of prongs of the surgical occluding device 200) and is inserted to the target tissue laparoscopically. Each arm of the firing device is positioned adjacent each location of the plurality of deflection locations 230a , 230b , 230c , 230d of the surgical occluding device 200. Actuation of the firing device causes the arms of the firing device to move radially inward, thereby causing the plurality of deflection locations 230a , 230b , 230c , 230d of the surgical occluding device 200 to move radially inward such that the surgical occluding device 200 transitions from its O-like orientation (FIG. 6) to its compressed orientation (FIG. 7). In aspects, the distal surgical occluding device 200 is compressed initially, followed by compression of the proximal surgical occluding 200. As shown in FIG. 7, when the surgical occluding devices 200 are in the compressed configuration, the tissue lumen “T” is also compressed into a corresponding shape.

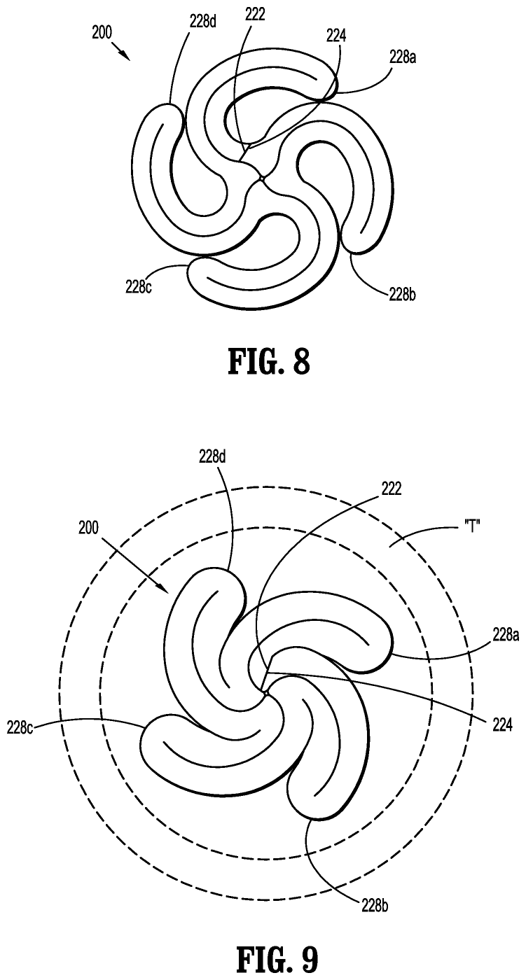

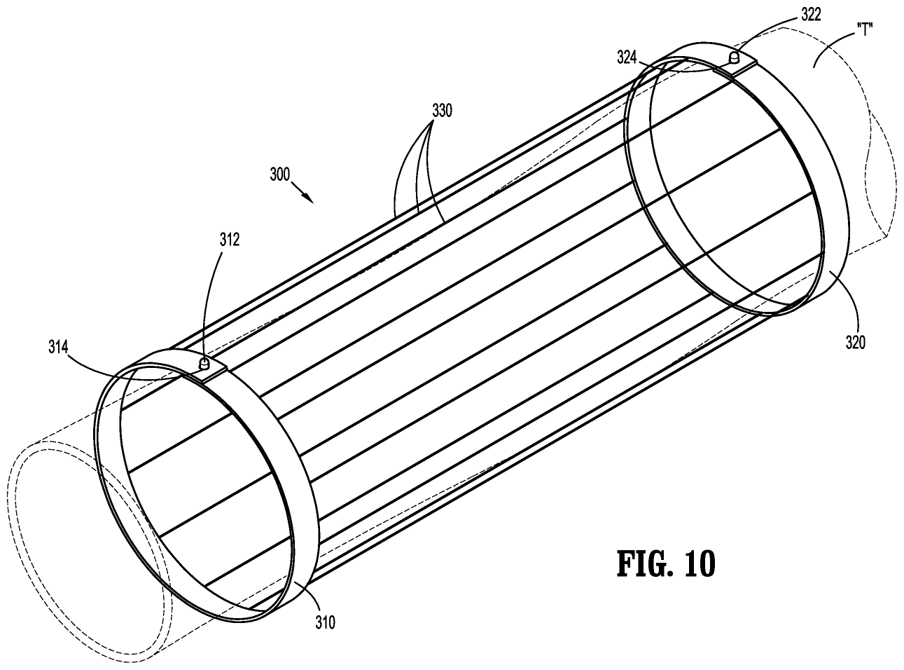

When the surgical occluding devices 200 are in the compressed configurations, the firing device (or another device) is used to rotate the plurality of prongs 228a , 228b , 228c , 228d of the body portion 220 of each surgical occluding device 200 about a central axis “A-A” (FIG. 5) of the surgical occluding device 200, which results in each surgical occluding device 200 having a curved orientation (FIG. 8). Continued rotation of the plurality of prongs 228a , 228b , 228c , 228d forms a tightly rolled surgical occluding device 200 and corresponding tissue lumen “T” (only the outer, non-rolled tissue lumen is shown in FIG. 9). Further, in aspects, the distal surgical occluding device 200 and the proximal surgical occluding device 200 are rotated in opposite directions from each other (i.e., one surgical occluding device 200 is rotated clockwise, and the other surgical occluding device 200 is rotated counterclockwise). Such a rotation in opposite directions may enable tightly rolling up the tissue lumen “T” (e.g., colon pleats) with minimal torque to adjacent tissue.

Following the rotation of the surgical occluding devices 200, a suture may be used to create a purse-string to help occlude the tissue lumen “T.” Next, the tissue between the surgical occluding devices 200 is cut, and, in aspects, the surgical occluding devices 200 are removed. Next, an anastomosis procedure may be performed.

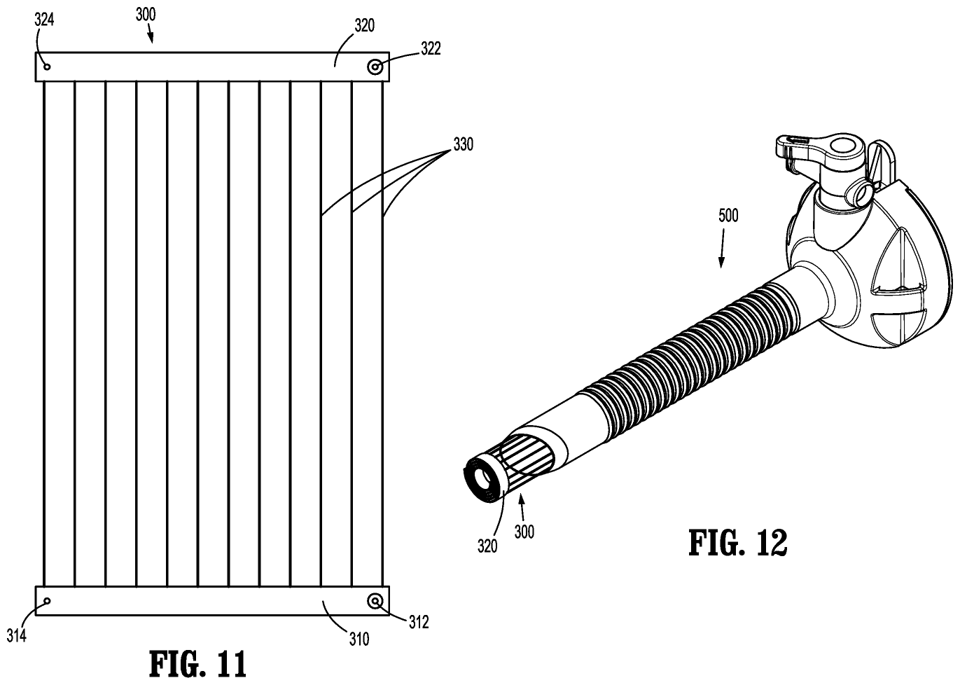

FIGS. 10-16 illustrate a third aspect of a surgical occluding device according to the disclosure, referenced generally as numeral 300. The surgical occluding device 300 is illustrated around a tissue lumen “T,” and includes a proximal band or ring 310, a distal band or ring 320, and plurality of spindles 330 interconnecting the proximal ring 310 and the distal ring 320. Each of the proximal ring 310 and the distal ring 320 is movable between an open, flat, rectangular orientation (FIG. 11), and a closed, circular orientation (FIG. 10). As the proximal ring 310 and the distal ring 320 move between the open orientation and the closed orientation, the surgical occluding device 300 moves between an open orientation (FIG. 11) and a closed orientation (FIG. 10).

With particular reference to FIG. 11, a first end of the proximal ring 310 includes a first engagement feature 312 (e.g., a post), and a second end of the proximal ring 310 includes a second engagement feature 314 (e.g., an aperture). The first engagement feature 312 of the proximal ring 310 is configured to engage (e.g., releasably engage) the second engagement feature 314 of the proximal ring 310. Likewise, a first end of the distal ring 320 includes a first engagement feature 322 (e.g., a post), and a second end of the distal ring 320 includes a second engagement feature 324 (e.g., an aperture). The first engagement feature 322 of the distal ring 320 is configured to engage (e.g., releasably engage) the second engagement feature 324 of the distal ring 320.

Each spindle of the plurality of spindles 330 is secured to both the proximal ring 310 and the distal ring 320. While twelve evenly-spaced spindles 330 are shown, the surgical occluding device 300 may include more or fewer spindles, and different spindle-spacing without departing from the scope of the disclosure.

In use, to deliver the surgical occluding device 300 to a desired location surrounding a tissue lumen “T,” the surgical occluding device 300 is rolled into a coiled configuration (i.e., having a smaller diameter than when in the closed orientation), and positioned within a distal portion of a cannula 500 (FIG. 12).

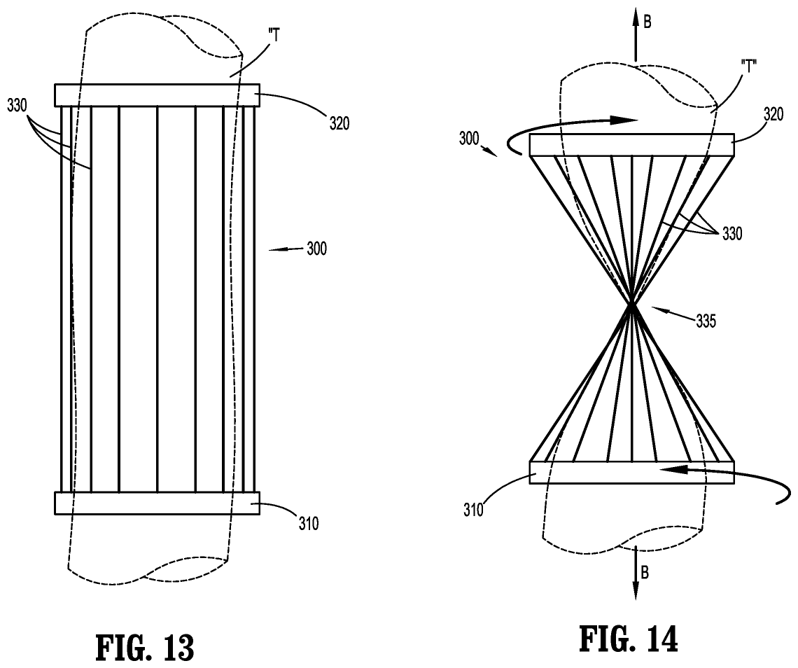

With the aid of the cannula 500, the surgical occluding device 300 is positioned adjacent the tissue lumen “T” and is flattened into a relatively flat shape. Surgical graspers or forceps may be used the flatten the surgical occluding device 300, and may also be used to wrap the surgical occluding device 300 around the tissue lumen “T,” manipulate the surgical occluding device 300 such that the first engagement feature 322 engages the second engagement feature 324, and such that the surgical occluding device 300 forms a cylindrical shape (see FIGS. 10 and 13).

After the surgical occluding device 300 is positioned around the tissue lumen “T” and transitioned to its closed orientation (FIGS. 10 and 13), a firing device is used to rotate the proximal ring 310 and the distal ring 320 of the surgical occluding device 300 about a central axis “B-B” of the surgical occluding device 300 (FIG. 14). More particularly, and with specific reference to FIG. 14, the proximal ring 310 and the distal ring 320 are rotated in opposite directions from each other (e.g., the distal ring 320 is rotated clockwise, and the proximal ring 310 is rotated counterclockwise). Such a rotation in opposite directions may enable tightly rolling up the tissue lumen “T” (e.g., colon pleats) with minimal torque to adjacent tissue. The rotation of the proximal ring 310 and the distal ring 320 in opposite directions from each other also causes the plurality of spindles 330 to twist, forming a narrow portion 335 (FIG. 14) at or near the longitudinal midpoint of the plurality of spindles 330. The twisting of the plurality of spindles 330 also provides hemostasis, as it causes the tissue lumen “T” to close.

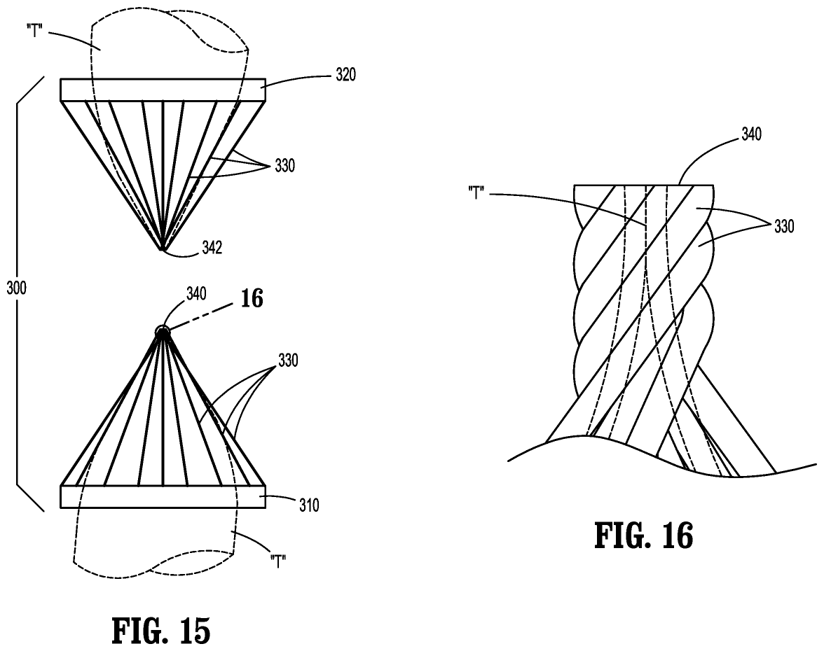

After the tissue lumen “T” is closed, the narrow portion 335 is cut using appropriate surgical clippers (FIG. 15), thereby creating a proximal cut line 340 (FIGS. 15 and 16) and a distal cut line 342 (FIG. 15). In aspects, the spindles 330 are formed from an appropriate material such that the spindles 330 maintain their twisted configuration after the narrow portion 335 is cut. In aspects, portions of the surgical occluding device 300 are then removed, and an anastomosis procedure may be performed.

While the above description contains many specifics, these specifics should not be construed as limitations on the scope of the disclosure, but merely as illustrations of various aspects thereof. Therefore, the above description should not be construed as limiting, but merely as exemplifications of various aspects. Those skilled in the art will envision other modifications within the scope and spirit of the claims appended hereto.

What is claimed is:

1. A surgical occluding device, comprising:
a body portion configured to radially surround a tissue lumen, the body portion including:
a plurality of segments; and
a plurality of folds, each fold of the plurality of folds is defined between two adjacent segments of the plurality of segments, wherein half of the folds of the plurality of folds define acute angles, and wherein half of the folds of the plurality of folds define obtuse angles;
wherein the body portion is movable from a first orientation where the acute angle defines a first acute angle, to a second orientation where the acute angle defines a second, smaller acute angle.

2. The surgical occluding device according to claim 1, wherein the plurality of segments includes ten segments.

3. The surgical occluding device according to claim 1, wherein when the body portion is in the first orientation, the obtuse angle defines a first obtuse angle, and wherein when the body portion is in the second orientation, the obtuse angle defines a second, smaller obtuse angle.

4. The surgical occluding device according to claim 1, further including a length of suture engaged with the body portion.

5. The surgical occluding device according to claim 4, wherein the length of suture is engaged with each segment of the plurality of segments.

6. The surgical occluding device according to claim 1, wherein the plurality of folds includes ten folds.

7. The surgical occluding device according to claim 1, wherein the body portion forms a star shape in its first orientation.

8. The surgical occluding device according to claim 7, wherein the body portion forms a star shape in its second orientation.

9. A surgical occluding device configured to radially surround a tissue lumen, comprising:
a proximal band including a first end and a second end;
a distal band including a first end and a second end; and
a plurality of spindles interconnecting the proximal band and the distal band,
wherein the proximal band is movable from a first orientation where the first end of the proximal band is free from contact with the second end of the proximal band to a second orientation where the first end of the proximal band contacts the second end of the proximal band, and wherein the distal band is movable from a first orientation where the first end of the distal band is free from contact with the second end of the distal band to a second orientation where the first end of the distal band contacts the second end of the distal band.

10. The surgical occluding device according to claim 9, wherein the first end of the proximal band includes structure that is configured to engage structure on the second end of the proximal band to retain the proximal band in the second orientation.

11. The surgical occluding device according to claim 10, wherein the first end of the distal band includes structure that is configured to engage structure on the second end of the distal band to retain the distal band in the second orientation.

12. The surgical occluding device according to claim 9, wherein the proximal band and the distal band are configured to be rolled into a coiled configuration to fit within a portion of a cannula.

13. The surgical occluding device according to claim 9, wherein the plurality of spindles includes between ten spindles and fourteen spindles.

14. The surgical occluding device according to claim 9, wherein the plurality of spindles includes twelve spindles.

15. The surgical occluding device according to claim 9, wherein each spindle of the plurality of spindles is evenly spaced from adjacent spindles of the plurality of spindles.

16. A method of occluding a tissue lumen, comprising:
positioning a first tissue occluding device around a distal portion of a tissue lumen, the tissue lumen defining a longitudinal axis;
positioning a second tissue occluding device around a proximal portion of a tissue lumen;
compressing the first tissue occluding device to move the first tissue occluding device from a C-shaped orientation to an O-shaped orientation;
compressing the second tissue occluding device to move the second tissue occluding device from a C-shaped orientation to an O-shaped orientation;
deflecting a plurality of locations of the first tissue occluding device to move the plurality of locations radially inward; and
deflecting a plurality of locations of the second tissue occluding device to move the plurality of locations radially inward.

17. The method according to claim 16, further comprising rotating the first tissue occluding device about the longitudinal axis.

18. The method according to claim 17, further comprising rotating the second tissue occluding device about the longitudinal axis.

19. The method according to claim 18, wherein the first tissue occluding device is rotated in a first direction, and wherein the second tissue occluding device is rotated in a second, opposite direction.

20. The method according to claim 16, wherein deflecting the plurality of locations of the first tissue occluding device includes deflecting four locations of the first tissue occluding device.
